(19) United States
(12) Patent Application Publication
(10) Pub. No.: US 2010/0143935 A1
(43) Pub. Date: Jun. 10, 2010

(54) c-KIT Phosphorylation in Cancer

(75) Inventor: Darren W. Davis, Houston, TX (US)

Correspondence Address: BAKER & MCKENZIE LLP, 711 Louisiana, Suite 3400, HOUSTON, TX, 77002, US

(73) Assignee: Apocell, Inc., Houston, TX (US)

(21) Appl. No.: 12/517,182

(22) PCT Filed: Dec. 3, 2007
(86) PCT No.: PCT/US2007/086317

§ 371 Date: Feb. 17, 2010

Related U.S. Application Data
(60) Provisional application No. 60/868,325, filed on Dec. 1, 2006.

(51) Int. Cl. G01N 33/53; C07K 16/00

(52) U.S. Cl. 435/7.1; 530/387.9; 530/388.1; 530/387.3

(57) ABSTRACT

An antibody is disclosed for the detection of phosphorylated c-KIT. A method of diagnosing and monitoring cancers responsive to treatment using an anti-phospho-c-KIT antibody are also disclosed. A diagnostic kit is also provided for the detection and monitoring of cancers responsive to tyrosine phosphorylation inhibitor treatment.

### PRIOR RELATED APPLICATIONS

This application claims the benefit of U.S. Provisional Application Ser. No. 60/868,325 filed Dec. 1, 2006 entitled “c-KIT Phosphorylation in Cancer,” incorporated by reference herein in its entirety.

### FEDERALLY SPONSORED RESEARCH STATEMENT

The present invention may have been developed with funds from the United States Government. Therefore, the United States Government may have certain rights in the invention.

### REFERENCE TO MICROFICHE APPENDIX

Not applicable.

### FIELD OF THE INVENTION

The invention relates to the diagnosis, treatment, and monitoring of cancer by detecting c-KIT phosphorylation at. Antibodies for detecting phosphorylation of c-KIT and kits for detecting phosphorylation of c-KIT are also provided.

### BACKGROUND OF THE INVENTION

Targeting cell signaling pathway in tumor cells has led to advances in treatment of many human malignancies. Imatinib is the first example of a tyrosine kinase inhibitor that blocks relevant oncogenic signaling pathways. It was originally developed by NOVARTIS PHARMACEUTICALS® as a specific inhibitor of the BCR-ABL tyrosine kinase, which arises from a t (9;22) chromosomal translocation (Philadelphia chromosome) in chronic myelogenous leukemia (CML). Imatinib competes for the ATP binding site on BCR-ABL, inhibiting its kinase activity leading to a complete response in most CML patients (Druker, 2001). Interestingly, Imatinib also inhibits other protein tyrosine kinases (PTKs) such as proto-oncogene c-KIT and the platelet-derived growth factor (PDGF) receptor (PDGFR). Recently, imatinib has shown success in treating gastrointestinal stroma tumors (GISTs) that harbor c-KIT mutants (van Oosterom, 2001; Druker, 2001; Kantarjian, 2002; Demetri, 2002). Imatinib has also been used to treat hypereosinophilic syndrome in which PDGFR plays a role in disease progression (Gleich, 2002; Cools, 2004). Therefore, imatinib is now considered a first-line agent for many malignancies with aberrant PTK signaling through ABL, c-KIT, and PDGFR.

Since all three known targets of imatinib (ABL, c-KIT, and PDGFR) are generally expressed in melanoma (Shen, 2003), research efforts have focused on testing the efficacy of imatinib in advanced melanoma. The c-KIT protein is a membrane receptor (−145 KD) with an intrinsic tyrosine kinase activity (Blume-Jensen, 1991). The ligand for c-KIT is stem cell factor (SCF). It was postulated that dimeric SCF molecules bind to two c-KIT monomers (Lemmon, 1997; Philo, 1996). The activated c-KIT becomes autophosphorylated on a number of tyrosine residues including Y703 and Y721 (Blume-Jensen, 1991; Duensing, 2004). These tyrosine residues are responsible for the activation of the major cancer survival pathway through phosphoinositide 3 kinase (PI3-K). These phosphorylated tyrosine residues serve as docking sites for Src homology 2 (SH2) or phosphotyrosine binding (PTB) domain-containing signal transduction molecules. In particular, the regulatory p85 subunit of PI3-K interacts with phospho-Y721 through its SH2 domain (Carpenter, 1993). In turn, the PI3-K is activated and the downstream effector AKT is phosphorylated/activated leading to inhibition of apoptosis. The importance of c-KIT and SCF signaling pathway has been demonstrated in normal melanocyte development (Grabbe, 1994; Nishikawa, 1991; Giebel, 1991). Expression of c-KIT has also been found on the membrane of a number of cancer cells including melanoma, colon carcinoma, small cell lung carcinoma, mast cell disease, testicular germ cell tumors, endometrial carcinomas, papillary and follicular thyroid carcinomas, small cell carcinomas, malignant melanomas, ovarian epithelial carcinomas, serous ovarian carcinoma, malignant melanoma, adenoid cystic carcinoma, salivary gland tumors, metastatic renal cell carcinoma (Arber, 1998; Heinrich, 2002; Mouriaux, 2003; Lefevre, 2004). Interestingly, a significant level of phosphorylated c-KIT was observed in uveal melanoma cell lines without activating mutations (Lefevre, 2004; All-Ericsson, 2004).

Since the PI3-K/AKT pathway is the major regulator of survival of many cancer cells including melanoma (Luo, 2003), imatinib has been considered to be a promising drug to treat melanoma and other tumors that depend on the c-KIT/PI3-K/AKT pathway for survival. It is therefore not surprising that some melanoma cells lines are sensitive to the growth inhibitory activity of imatinib (Lefevre, 2004; All-Ericsson, 2004). Activating mutations of c-KIT have been found in human GISTs (van Oosterom, 2001; Druker, 2001; Kantarjian, 2002; Demetri, 2002), but have not been well characterized in other human cancers. Alternative mRNA slicing has been shown to be responsible for several c-KIT isoforms, e.g., the absence of a tetrapeptide sequence (GNNK) (Reith, 1991; Crosier, 1993); the absence of a single serine residue in the kinase insert region (Δser) (Crosier, 1993); and the truncated c-KIT containing only the partial kinase domain (tr-kit) (Rossi, 1992). However, the link between these spliced variants and tumorigenesis is unclear. Phase II trials in metastatic melanoma have not been encouraging either, and clinical research has been unable to identify a correlation between c-KIT mutation, splice variants, or truncations and metastatic melanoma (Wyman, 2006; Eton, 2004).

In summary, the involvement of c-KIT in tumor progression is complex and varies between different types of cancer. This phenomenon underscores the importance of identifying a group of patients who are most likely to respond to treatment with phospho-tyrosine inhibitors such as imatinib or sunitinib. Currently there is no commercial assay that can predict responsiveness to targeted therapy based on KIT phosphorylation. What is needed is a predictive assay that will identify cancer patients that will respond to treatment with specific tyrosine kinase inhibitors, methods of monitoring tyrosine kinase activity during treatment, and kits for detecting tyrosine phosphorylation either directly from tumor biopsies or in blood samples.

### SUMMARY OF THE INVENTION

Predictive biomarkers for therapeutic response to cancer therapy, methods of diagnosing cancer, and methods of monitoring treatment phosphorylation inhibitors are disclosed herein. Specifically c-KIT phosphorylation at Y721 is a marker for treatment response in patients with specific phosphorylation of c-KIT Y721, antibodies for detecting phosphorylation at Y721, and methods of predicting or monitoring cancer treatment and response to tyrosine phosphorylation inhibitor therapy are disclosed. In one embodiment, hyperphosphorylation of c-KIT at Y721 in acral lentinginous melanoma patients predicts clinical response to imatinib. In another embodiment, phosphorylation of c-KIT at Y721 is inhibited in GIST patients who respond to sunitinib. Y703 and other phosphorylated residues may also be monitored for hyperphosphorylation. Additionally hyperphosphorylated residues Y721 and/or Y703 can be monitored with respect to total c-KIT.

Methods of predicting cancer response in a patient: isolating a cancer sample from a patient; adding an antibody specific for phosphorylated c-KIT; detecting binding of said antibody to phosphorylated c-KIT in said sample; and identifying said cancer sample as a cancer responsive to treatment with an inhibitor of c-KIT phosphorylation.

Methods of predicting cancer response to treatment: isolating a blood sample from a patient; adding an antibody specific for phosphorylated c-KIT; detecting binding of said antibody to phosphorylated c-KIT in said sample; and identifying said patient as having a cancer responsive to treatment with an inhibitor of c-KIT phosphorylation.

Methods of monitoring patient response to cancer treatment: isolating a sample from a patient with a cancer; adding an antibody specific for phosphorylated c-KIT; detecting binding of said antibody to phosphorylated c-KIT in said sample; repeating the steps after one or more treatments; and determining response of to cancer treatment where a decrease in antibody binding indicates a remission of said cancer.

Additionally, an antibody that binds to a non-phosphorylated epitope of c-KIT can be used to determine levels of total c-KIT. By comparing phosphorylated c-KIT to total c-KIT levels, one determines the amount of phosphorylated c-KIT in the sample. Further DNA fragmentation can be measured using a TUNEL assay to correlate reduced phosphorylation of c-KIT with apoptosis.

A kit is described for the detection of cancers susceptible to tyrosine kinase inhibitors, the kit contains: an antibody specific for phosphorylated c-KIT; a detection antibody that binds the antibody specific for phosphorylated c-KIT; and a detection reagent that specifically reacts with the detection antibody. The kit may also have a control sample containing known samples of c-KIT phosphorylation.

Cancer includes solid tumor, blood based cancer, metastatic cancer, sarcoma, or melanoma, gastrointestinal cancer, breast cancer, leukemia, lymphoma, lung cancer, acral lentiginous melanoma, renal cell carcinoma, colon carcinoma, small cell lung carcinoma, mast cell disease, testicular germ cell tumors, endometrial carcinomas, papillary and follicular thyroid carcinomas, small cell carcinomas, malignant melanomas, ovarian epithelial carcinomas, serous ovarian carcinoma, malignant melanoma, adenoid cystic carcinoma, salivary gland tumors, and metastatic renal cell carcinoma.

Phosphorylation specific c-KIT antibodies may be directed to and phosphorylated c-KIT residue including tyrosine 703 or tyrosine 721. Phosphorylation specific c-KIT antibodies may be any antibody that binds to a single epitope including monoclonal antibody, recombinant single chain antibody and site specific protein-nucleic acids (PNAs). An antibody that binds specifically to phosphorylated c-KIT at a single epitope and does not bind to non-phosphorylated c-KIT is preferred. In another embodiment a polyclonal, monoclonal, recombinant, or synthetic PNA antibody that binds specifically to phosphorylated Y721 is used to monitor c-KIT hyperphosphorylation. Additionally a polyclonal, monoclonal, recombinant, or synthetic PNA antibody that binds specifically to phosphorylated Y703 may be used to monitor c-KIT hyperphosphorylation.

A detection system may be any detection antibody and marker including fluorescence, peroxidase, radioactive, silver staining, colloidal gold, and the like. In one example, the detection antibody is an anti-mouse antibody conjugated with horseradish peroxidase and the detection reagent is a DAB substrate buffer. In another embodiment, the detection antibody is anti-mouse antibody conjugated with a fluorescent probe and said detection reagent is a chemiluminescent immunoassay (CLIA). The optional control sample can be a slide containing high, basal, and background levels of phosphorylated c-KIT.

The human c-KIT protein is also known as proto-oncogene tyrosine-protein kinase (KIT or c-KIT) and stem cell growth factor receptor precursor (SCFR). More information about the c-KIT protein may be found at NCBI's GENBANK® database. A summary of c-KIT protein variants is provided in Table 1.

TABLE 1
KIT Protein Variants

| GenBank | | | | |
|---|---|---|---|---|
| Acc # | Protein | Length | % AA ID | Ref |
| NP_000213 | KIT | 976 aa | 976/976 (100%) | Duronio, 1992 |
| AAC50968 | KIT | 976 aa | 976/976 (100%) | Andre, 1992 |
| AAH71593 | KIT | 976 aa | 975/976 (100%) | Strausberg, 2002 |
| AAC50969 | KIT | 972 aa | 972/976 (99%) | Andre, 1992 |
| CAD27356 | KIT | 160 aa | 160/161 (99%) | Andersson, 2002 |
| AAB29301 | KIT | 54 aa | 29/54 (53%) | Toyota, 1994 |

The terms “complementary” and “complement”, as used herein refer to polynucleotide sequences that are capable of base pairing with contiguous polynucleotide sequences due to sequence homology throughout the complementary regions. Various lengths of DNA will hybridize based on % homology, GC content, and annealing conditions. Hybridization may be observed with greater than 80%, 85%, 90%, 95%, or 99% homology. Sequences with 100% homology are an exact complement.

As used herein “recombinant” is relating to, derived from, or containing genetically engineered material. Recombinant DNA can be carried on a vector or integrated into the chromosome of the host cell. Many vectors are known which can be used in a variety of species. Stable chromosomal integration methods are also well documented.

In calculating “% identity” unaligned terminal portions of a query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova T A & Madden T L (1999) FEMS Microbiol. Lett. 174:247-250. The default parameters were used, except the filters were turned OFF. As of Dec. 1, 2006 the default parameters were as follows: BLASTN or BLASTP as appropriate; Matrix=none for BLASTN, BLOSUM62 for BLASTP; G Cost to open gap default=5 for nucleotides, 11 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=11 for nucleotides, 3 for proteins; y dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (www.ncbi.nlm.nih.gov/BLAST/).

Common restriction enzymes and restriction sites are found at NEB® (NEW ENGLAND BIOLABS®, www.neb.com) and INVITROGEN® (www.invitrogen.com) as well as other commercial enzyme suppliers. ATCC®, AMERICAN TYPE CULTURE COLLECTION™ (www.atcc.org), DSMZ®, DEUTSCHE SAMMLUNG VON MIKROORGANISMEN UND ZELLKULTUREN™ (www.dsmz.de), KBIF®, KOREAN BIOLOGICAL RESOURCE CENTER™ (kbif.kribb.re.kr), and WDCM®, WORLD DATA CENTRE FOR MICROORGANISMS™ (wdcm.nig.ac.jp) have extensive collections of cell strains that are publicly available. NEB®, INVITROGEN®, ATCC®, DSMZ®, KBIF®, and WDCM® databases are incorporated herein by reference.

Imatinib is also known as imatinib mesylate, GLIVEC™, GLEEVEC™, STI571, and 4-((4-methyl)-1-piperazinyl)methyl)-N-(4-methyl-3-((4-(3-pyridinyl)-2-pyrimidinyl)amino)phenyl)-benzamide. More information about imatinib is available from NCBI's PubChem CID # 5291 and CAS Reg # 152459-95-5, incorporated herein by reference. Imatinib variants, polymorphs, conjugates, enantiomers, and derivatives are described in U.S. Pat. Nos. 5,521,184 and 7,300,938, incorporated herein by reference and commercially available from a variety of sources.

Sunitinib is also known as sunitinib malate, SUTENT™, SU-11248, and N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide. More information about sunitinib is available from NCBI's PubChem CID # 5329102 and CAS Reg # 326914-13-0, incorporated herein by reference. Sunitinib variants, conjugates, enantiomers, and derivatives are described in U.S. Pat. Nos. 6,573,293, 6,677,368, and 7,125,905 incorporated herein by reference, and is commercially available from a PFIZER®.

An antibody is an immunoglobulin, a specialized immune protein, produced by the introduction of an antigen or immunogen into the body. An antibody possesses the remarkable ability to bind the antigen that triggered its production. Antibodies are produced by B lymphocyte of animals injected with antigens such as foreign proteins.

Polyclonal antibodies are produced by a number of different cell types. Polyclonal antibodies are typically isolated from animals treated with antigen to induce immune response.

Monoclonal antibodies, abbreviated mAb or moAb, are antibodies that are identical because they are produced by one type of immune cell that are all clones of a single parent cell.

A hybridoma is a hybrid cell, composed of a B lymphocyte fused to a tumor cell, which grows indefinitely in tissue culture and is selected for the secretion of a specific antibody of interest.

A single-chain antibody (SCA) is a recombinant protein encoding the binding portion of a monoclonal antibody. The SCA can be fused to functional domains, like streptavidin, peroxidase, a His-tag, or other protein to provide a more functional and easily detected protein. Because the SCA is a recombinant protein it can be consistently produced in large quantities.

Specific antibody binding is a single binding immunoglobulin or fragment thereof that binds to a single epitope. A phospho-721 c-KIT specific antibody is a uniform antibody preparation containing an monoclonal, single-chain antibody fragment, a phospho-721 c-KIT binding peptide-nucleic acid, and the like that bind uniquely the phosphorylated c-KIT 721.

### BRIEF DESCRIPTION OF THE DRAWINGS

FIG. 1. Clinical and radiological studies of a partial response to imatinib. A near-complete response to imatinib of a melanoma patient. All metastatic lesions shrank. (A) Response of in-transit metastases on the right thigh. (B) Computed tomography scan showing response of left external iliac lymph node (arrow). (C) Positron emission tomography scans showing decrease in fluorodeoxyglucose uptake in all lesions (arrows).

FIG. 2. The effect of Imatinib on the phosphorylation of c-KIT at Y721 in the melanoma patients. Tumor biopsies were stained by anti-phospho-Y721 c-KIT antibody followed by secondary antibody conjugated with a fluorescence probe. Laser scanning cytometry (LSC) was used to analyze the medium fluorescence intensity (MFI) across the tumor biopsies. The MFI in tumors collected before treatment (Pre-treatment) (blue bars) and 2 weeks after treatment (Post-treatment) (purple bars) are indicated.

FIG. 3. Imatinib inhibits the phosphorylation of c-KIT at Y721 in the responder. (Top panels), Tumor biopsies obtained from the responder at baseline and 2 weeks after treatment (Post-treatment) were stained by anti-phospho-Y721 c-KIT antibody followed by secondary antibody conjugated with a fluorescence probe. Representative images are shown. The tumor sample stained only by the secondary antibody serves as a negative control. (Bottom panels), LSC was used to analyze the intensity of the phospho-Y721 of c-KIT (Cy5) across the entire cross section of tumor biopsies. The relative intensity is shown in 3-dimensional tissue map. The levels of protein expression are indicated by the number, height, and color of bars. Note that the expression of phospho-Y721 is homogenous in the baseline biopsy, whereas the imatinib-treated biopsy shows fewer bars and lower intensity of the expression of Y721 phosphorylation. (The level of intensity is indicated by color: red>green>blue.)

FIG. 4. Imatinib induces tumor and endothelial cell apoptosis in Responder. Tumor biopsies obtained from the responder at baseline (Pre-treatment) and 2 weeks after treatment (Post-treatment) were stained by anti-CD31 antibody followed by secondary antibody conjugated with a fluorescence probe (red). The stained samples were further analyzed for apoptotic cells by TUNEL assay (green). Representative images are shown. The corresponding CD31 and TUNEL images are merged (Composite).

### DESCRIPTION OF EMBODIMENTS OF THE INVENTION

Hyperphosphorylation of c-KIT at Y721 can serve as a predictive marker for cancer treatment with tyrosine phosphorylation inhibitors response in patients with a variety of cancers. By understanding the mechanism underlying c-KIT hyperphosphorylation, additional markers will be identified to better predict response in cancer patients. By validating the c-KIT phosphorylation at Y721 can be used to identify a subgroup of cancer patients most likely to respond to tyrosine phosphorylation inhibitors. A commercial kit (C-KIT PHOSPHORYLATION TEST KIT™) may be developed to identify patients for treatment with tyrosine phosphorylation inhibitors. Cancers responsive to tyrosine phosphorylation inhibitors include melanoma, colon carcinoma, small cell lung carcinoma, mast cell disease, testicular germ cell tumors, endometrial carcinomas, papillary and follicular thyroid carcinomas, small cell carcinomas, malignant melanomas, ovarian epithelial carcinomas, serous ovarian carcinoma, malignant melanoma, adenoid cystic carcinoma, salivary gland tumors and the like that have high levels of c-KIT Y271 phosphorylation.

In one embodiment c-KIT phosphorylation at Y721 serves as a marker for imatinib responsiveness in patients with acral lentinginous melanoma. In another embodiment, c-KIT phosphorylation at Y721 serves as a marker for sunitinib treatment in patients with gastrointestinal stroma tumors (GIST). The examples demonstrate that c-KIT phosphorylation can be used as a predictive marker for cancers that will respond to tyrosine phosphorylation inhibitors.

### Example 1

### Clinical Analysis of Imatinib Treatment

Melanomas express the major targets of imatinib such as c-KIT and PDGFRs and are considered to be a suitable disease for imatinib treatment. Unfortunately, results obtained from the clinical trials have been disappointing (Wyman, 2006; Eton, 2004). One Phase II trial with high dose imatinib (800 mg/day) involving 26 patients from Vanderbilt-Ingram Cancer Center and Beth Israel Deaconess Medical Center showed significant toxicity but failed to show any clinical response (Wyman, 2006). Low levels of c-KIT and other imatinib targets were observed in these tumors, and that may explain in part a lack of clinical response in these patients.

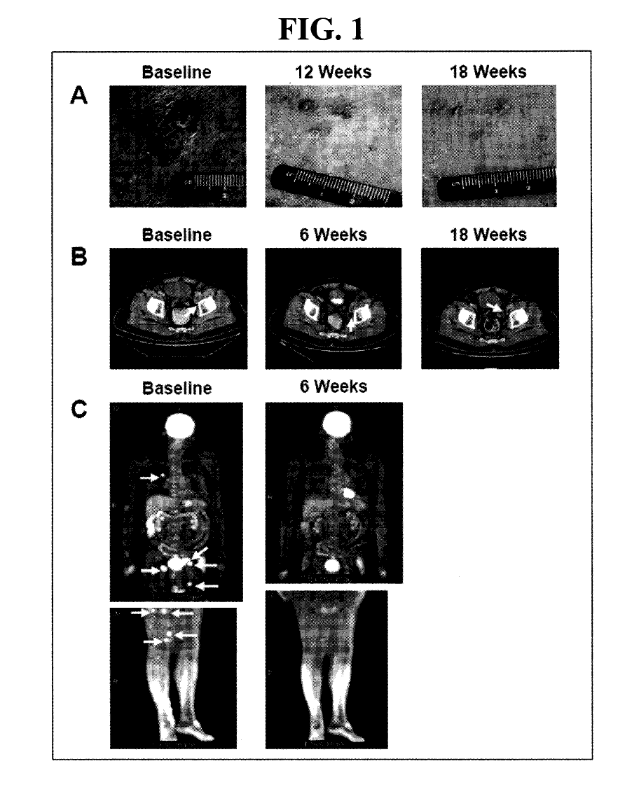

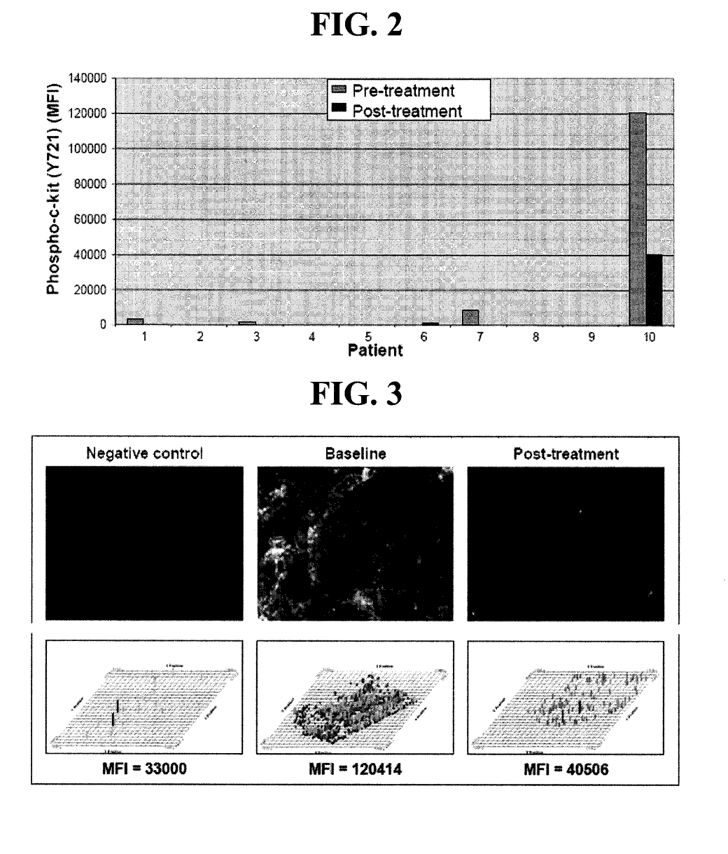

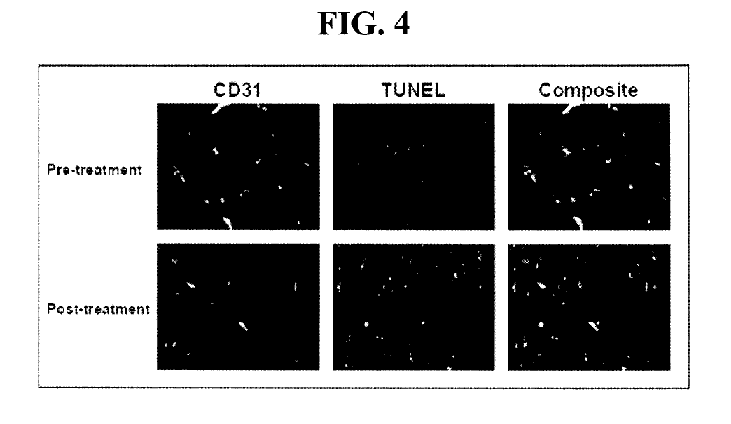

In a second Phase II trial conducted at M. D. Anderson Cancer Center, 21 patients with stage III (10%) and IV (90%) melanoma were enrolled. All had tumors expressing at least one target PTK, i.e., c-KIT, PDGFRα, or PDGFRβ. These patients received a total of 33 courses of imatinib (median, one course per patient; range, one to nine). Imatinib was administered orally at a dose of 400 mg twice a day. Twenty of the twenty one patients observed (95%), had progressive melanoma over a 12 week period. However, one patient (5%) with acral lentiginous melanoma had a near-complete response that lasted more than a year (FIG. 1). This one patient tolerated nine consecutive courses of treatment over a 1 year period. This patient was a 66-year-old man who had a near-complete response to imatinib in numerous metastases of the cutaneous and subcutaneous tissues, inguinal and iliac lymph nodes, and lungs. LSC was used to determine protein phosphorylation levels. This patient had the highest levels of c-KIT Y721 phosphorylation (FIGS. 2 and 3). The levels of phospho-Y721 were significantly reduced after imatinib treatment in the responder (FIG. 3). We also found higher levels of apoptosis in both endothelial and tumor cells in the responder when compared with non-responders (FIG. 4). Inhibition of phospho-Y721 of c-KIT in this patient's tumor correlated well with the clinical response.

Serial physical exams with photographs confirmed gradual depigmentation and complete resolution of all palpable skin nodules (FIG. 1A) and follow-up CT scans showed near complete resolution in nodal and lung metastases after 6 months. Follow-up computed tomography scans did not confirm ongoing major response in this patient until after twelve weeks of treatment (FIG. 1B). After just six weeks of treatment, a positron emission tomography (PET) scan revealed a marked reduction of metabolic activity in all disease sites (FIG. 1C). Hyperphosphorylation of c-KIT at Y721 in this patient provides a predictive marker for imatinib response in patients with acral lentiginous melanoma.

### Example 2

### Mechanism for Imatinib Responsiveness in Melanoma

The mechanism of Y721 hyperphosphorylation in the imatinib responder provides a method to develop novel treatments and monitor disease remission. Although it is known that both Y703 and Y721 are autophosphorylated by c-KIT kinase upon SCF stimulation (Duensing, 2004), the preferential phosphorylation of Y721 in the responder suggests a novel mechanism of c-KIT activation. To elucidate the mechanism, we use (1) RT-PCR and LSC analysis to examine the existence of aberrant c-KIT mRNAs and proteins; and (2) in vitro assays to examine the function of c-KIT mutant. Identification of a novel mechanism for c-KIT activation in tumors provides additional markers to predict responsiveness to imatinib in melanoma patients as well as patients with other types of cancer.

Phosphorylation status of imatinib targets, i.e., c-KIT, PDGFRα, and PDGFRβ were monitored. For c-KIT phosphorylation, tumor biopsies obtained at baseline (pre-treatment) and during the second week of treatment (post-treatment) were assayed. Since the phosphorylation of tyrosine 721 is critical in the c-KIT-mediated survival pathway (Serve, 1995; Carpenter, 1993), we tested whether or not the phosphorylation of Y721 is involved in the imatinib responsiveness in the responder. As shown in FIG. 2 and Table 2, at baseline, the phospho-Y721 of c-KIT is drastically higher (77-fold) in the responder (patient #10) than the average of the non-responders (n=9). These observations suggest a strong link between the hyperphosphorylation of Y721 of c-KIT and the clinical response to imatinib. We also examined the levels of phospho-Y703. The responder exhibited 36-fold higher levels of phospho-Y721 than that of the non-responder (Table 2). Moreover, we found that the levels of phosphor-PDGFRα or -β in the non-responders are higher than that of the responder. These data suggest that the c-KIT-mediated oncogenic pathway is the driving force for the tumorigenesis of melanoma in the responder.

TABLE 2
The phosphorylation status of c-KIT, and PDGFRα/β
in the melanoma patients.

| | Phospho- | Phospho- | | |
|---|---|---|---|---|
| | c-KIT | c-KIT | Phospho- | Phospho- |
| Clinical Outcome | (Y721) | (Y703) | PDGFRα | PDGFRβ |
| Non-responder | 1570 ± | 1130 (n = 1) | 770304 ± | 724938 ± |
| | 2966 | | 271269 | 17561 |
| | (n = 9) | | (n = 10) | (n = 10) |
| Responder (n = 1) | 120414 | 40465 | 29710 | 45759 |
| Fold difference | 77 | 36 | 30 | 16 |

Since activating mutations of c-KIT have been correlated with the imatinib responsiveness in GISTs (Joensuu, 2001; Tuveson, 2001), we tested whether mutations in c-KIT contribute to the hyperphosphorylation of Y703 or Y721 and responsiveness to imatinib in the responder. We sequenced all 21 exons of c-KIT allele using genomic DNA isolated from the tumor biopsy of the responder.

No mutations in the c-KIT gene coding sequence (data not shown).were identified. This result suggests that mechanism other than genomic mutation is responsible for the hyperphosphorylation of c-KIT at Y703 or Y721. Interestingly, the specimen from the responder did have an alternative splice site in exon 15 of the c-KIT kinase II domain. The deletion of a serine residue encoded by codon 715 at this site resulted in a short isoform of c-KIT (data not shown). Although interesting, the novel splice variant is not specific for the responder because 4 non-responding patients also expressed this alternatively spliced c-KIT mRNA in their tumors.

TABLE 3
Inhibition of c-KIT Phosphorylation in
GIST Patients Treated with Sunitinib.

| | Phospho-c-KIT | Phospho-c-KIT |
|---|---|---|
| Clinical Outcome | (Y721) | (Y703) |
| Responder | 94% decrease | 57% decrease |
| NON-Responder | 117% increase | 45% increase |

mRNA and Protein Analysis: Total RNA is isolated from tumor biopsies of one or more responder and non-responder patients. The c-KIT cDNAs are generated by RT-PCR technique followed by gel electrophoresis to separate DNA fragments. A wild type c-KIT cDNA (˜5 Kb) is used as a positive control. The unique bands present in the responder but not in the non-responders are excised from the gel and subcloned into plasmid vectors. DNA sequencing is performed on these unique clones to determine the abnormal mRNA species. Once the regions of abnormality are determined, appropriate antibodies are used to stain tumor biopsies from the responder and non-responders followed by LSC analysis to confirm the expression of the mutant c-KIT proteins in the responder's tumor but not in the non-responders' tumors. For example, if c-KIT is a deletion mutant in the responder, the antibody recognizes the deleted region will be negative in the responder's tumor but positive in the non-responders' tumors. Whereas, an antibody that recognizes total c-KIT is positive for both tumors. Western blot analysis is performed using lysates isolated from tumor biopsies to confirm the expression of these mutant c-KIT proteins.

Once a responder-specific c-KIT mutant is identified, the functional relationship between the mutant and imatinib responsiveness is confirmed. Stable clones that constitutively express c-KIT mutants are generated. Briefly, human melanoma cell line WM-266-4 (ATCC # CRL-1676™) that does not express c-KIT (Huang, 1998) is transfected with an empty vector, wild type c-KIT, and c-KIT mutants. A Neomycin resistant gene is co-transfected followed by G418 selection. The G418-resistant clones are screened for the expression of phospho-Y721 c-KIT and total c-KIT by Western blot analysis. The clones expressing hyperphosphorylated Y721 of c-KIT are chosen and treated with increasing dose of imatinib (0-15 μM) followed by the below assays to measure:
C-KIT phosphorylation: Western blot analysis of cells expressing mutant or wild type c-KIT using anti-phospho-Y703 or Y721 and anti-c-KIT antibodies.
Proliferation: Trypan-blue exclusion assay, MTT (3-(4,5-Dimethylthiazol-2-yl)-2,5-diphenyltetrazolium bromide) assay, and Double Thymidine block assay.
Apoptosis: DNA fragmentation measured by TUNEL assay and gel electrophoresis; and Poly-ADP-ribose polymerase (PARP) cleavage assay.
Cell cycle distribution: Propidium iodide stained cells are analyzed based on DNA content using flow cytometric analysis. Sub-G1 and S-phase cell populations are indicative of the apoptotic cells and proliferating cells, respectively.

In the case that the responder expressed aberrant c-KIT mutant with hyperphosphorylation at Y703 or Y721, we expect the stable cell lines expressing this c-KIT mutant will have higher level of c-KIT phosphorylation at Y721 than those expressing wild type c-KIT. Stable cell lines expressing mutant c-KIT are expected to be more sensitive to Imatinib-induced growth inhibition and apoptosis. Once identified, a c-KIT mutant that is unique to responders will be used to establish at least two stable cell lines expressing this c-KIT mutant that is sensitive to imatinib-induced growth inhibition and apoptosis.

It is possible that certain truncated mRNAs caused by alternative slicing may not be easily identified using gel electrophoresis. In that case, we will subclone all the c-KIT fragments followed by DNA sequencing to search for subtle mutations. If c-KIT gene remained wild type in the responder, the subsequent PK/PTP cDNA microarray may identify PK and/or PTP genes that are differentially expressed to correlate with hyperphosphorylation of Y721 in the responder. In the case that no difference in the PK/PTP expression profiles between the responder and the non-responders, it is possible the PK and PTP may be activated through the differential protein modification and not by gene expression. If so, we will identify the activated PK and PTP by performing Western blots using anti-phospho-tyrosine, -threonine, and -serine antibody cocktail. To identify these differentially modified proteins, the protein bands will be excised from the gel followed by mass spectrometry analysis (CTL BIO SERVICES®, Rockville, Md.).

However, it is possible that the responder expresses wild type c-KIT mRNAs or a splice variant not specific to the responder. Other possible mechanisms include the overexpression of protein kinase(s) (PK) and/or down regulation of protein phosphatase(s) (PTP) specific to Y721. To identify the candidate PK and/or PTP, we will employ the PK- and PTP-specific human DNA microarrays (JIVAN BIOLOGICS®, Inc. Berkeley, Calif.). This array profiles the expression of 528 PK genes and 231 PTP genes. Briefly, total RNA will be isolated from the responder's tumor biopsy and from a non-responder' tumor biopsy. RNA will then be converted into cDNAs in a standard thermal cycler. The cDNAs generated from the responder and the non-responder will be conjugated to different fluorescence probes, e.g., Cy5 and Cy3. Equal amount of labeled cDNAs from each sample will be mixed and hybridized the DNA microarrays using a standard hybridization kit (JIVAN BIOLOGICS®). The hybridization signals on the microarray will be analyzed using a software program (JIVAN BIOLOGICS®).

Once the candidate genes are identified and confirmed in the tumor biopsies of responders and non-responders, the expression of these genes is confirmed in stable cell lines expressing wild type and mutant c-KIT by Western blot and Northern blot analysis. For example, if a PK gene is overexpressed in the responder, stable cell lines expressing PK are generated by stably transfecting the PK gene into a human melanoma cell line, MeWo (ATCC # HTB-65™), which is known to express wild type c-KIT (Huang, 1998). The stable MeWo cell lines expressing PK are analyzed for c-KIT phosphorylation at Y721 and compared with the empty vector control cells. The proliferation, apoptosis, cell cycle distribution in response to imatinib is analyzed to confirm a functional relationship between this PK gene and c-KIT phosphorylation at Y721.

In contrast, if a PTP gene is down regulated in the responder, PTP knock down stable MeWo cell lines are established by stably transfecting a vector expressing PTP-specific siRNA into MeWo cells. Once the stable cell lines are verified for PTP knock down, the stable cell lines are analyzed for c-KIT phosphorylation at Y721 and compared with the empty vector control cells. The proliferation, apoptosis, cell cycle distribution in response to imatinib is analyzed to confirm a functional relationship between PTP gene knock-down and c-KIT phosphorylation at Y721.

We expect to identify the specific PK genes responsible for the phosphorylation of Y721 present or overexpressed in the responder but absent or down regulated in the non-responders. On the other hand, we expect to identify the specific PTP genes responsible for de-phosphorylating Y721 absent or down regulated in the responder but present or overexpressed in the non-responders. In either case, we expect the stable cell lines expressing PK or with PTP knock down will have higher phosphorylation of c-KIT at Y721 and higher proliferating rate than that of the empty vector control cells. We also expect these stable cell lines to be more sensitive to imatinib-induced growth inhibition and apoptosis than the control cells.

### Example 3

### Correlation of c-KIT Phosphorylation and Imatinib Responsiveness

In the previous study, we found one melanoma patient (n=40) with a dramatic near-complete response to imatinib treatment for more than a year. The most correlative marker for imatinib response is the hyperphosphorylation of c-KIT at Y721 in the responder at baseline. In addition, among this cohort, this patient is the only one with acral lentiginous melanoma. The goal of this aim is to validate the correlation between c-KIT hyperphosphorylation at Y703 or Y721 and patients with acral lentiginous melanoma. We hypothesize that patients with acral lentiginous melanoma have hyperphosphorylated c-KIT. To test this hypothesis, we will determine the phosphorylation status of c-KIT in the tumor biopsies obtained from acral lentiginous melanoma patients (n=5) using LSC. In addition, the expression of PK and/or PTP genes identified in Aim 1 will be likewise analyzed in these tumor biopsies. A successful demonstration of c-KIT hyperphosphorylation is unique in acral lentiginous melanoma patients will be a very important step forward to identify this group of patients as most likely candidates benefited for imatinib treatment.

The responder also showed a significant inhibition of phosphorylation of c-KIT at Y721 after imatinib treatment. Consistent with the inhibition of c-KIT-mediated survival pathway, the responder's tumor showed a substantial increase of the apoptotic endothelial and tumor cells after only 2 weeks of imatinib treatment, based on the CD31-TUNEL dual immunofluorescence studies (FIG. 4). Such an increase was not seen in the nonresponding patients' tumors (data not shown).

We will stain tumor biopsies obtained from patients with acral lentiginous melanoma (n=5) and non-responders with mucosal melanoma (n=5) (all tumor biopsies will be provided by our collaborator, Kevin Kim, M.D., M. D. Anderson Cancer Center) with anti-phospho-c-KIT (Y721) or (Y703) antibody. Total c-KIT protein will be stained in the same slides using anti-c-KIT antibody. To enable simultaneous measurement of both phospho-c-KIT and total c-KIT proteins, we will use two fluorescent probes whose emission spectra do not overlap. Briefly, the secondary antibody recognizes anti-phospho-c-KIT (Y721) or (Y703) antibody will be labeled with a fluorescent probe, e.g., Cy5 with emission max at 670 nm; whereas the secondary antibody recognizes the c-KIT antibody will be labeled with another probe, e.g., phycoerythrin (PE) with emission max at 578 nm. LSC will be used to simultaneously measure both the Cy5 and PE signals on a continuous scale across the tumor regions. The data will be analyzed using a software program to measure the mean fluorescence intensity (MFI) of phospho-c-KIT and total c-KIT staining.

We expect to see that c-KIT is hyperphosphorylated at Y703 or Y721 in the tumor biopsies from acral lentiginous melanoma but not in the other type of melanoma such as mucosal melanoma. In addition, we expect that the phosphorylation status of Y703 is comparable between acral lentiginous melanoma and that of the non-responders. The total c-KIT protein expression is expected to remain constant in all tumors. C-KIT levels may be measured using another antibody that does not bind phospho-Y703 or Y721. By monitoring the ratio of phospho-Y703 and/or Y721 to total c-KIT levels, the level of hyperphosphorylation may be directly monitored.

Once 5 acral lentiginous melanomas and 5 non-responder melanoma tumor samples are screened for phospho-Y721 and -Y703 of c-KIT, as well as the total c-KIT levels, a significant correlation between the markers and acral lentiginous melanomas (p<0.05) is expected.

It is possible that c-KIT phosphorylation at Y721 is not present in 100% of the tumor samples of acral lentiginous melanoma patients. In that case, we will include the expression of PK and/or PTP identified above in conjunction with Y721 phosphorylation to test if the correlation between these markers and acral lentiginous melanoma exists. Alternatively, we will increase the size of acral lentiginous melanoma cohort from 5 to 20 and repeat the above experiments to test whether or not the correlation between c-KIT phosphorylation at Y721 (with or without PK/PTP) and acral lentiginous melanoma is significant.

### Example 4

### Monoclonal Antibody for Predicting Responsiveness

We have used polyclonal antibodies to detect phospho-Y721 of c-KIT. Despite the clear technical and economical advantages for producing large quantities of polyclonal antibodies, monoclonal antibodies have certain advantages over polyclonal antibodies. Because of their immortal nature, hybridoma cells can be frozen, thawed, and re-cultured in vitro. As a result, for a given monoclonal line, there will be a homogeneous and renewable source of identical antibodies specific for the phospho-Y721 of c-KIT. This feature is especially important for commercializing an antibody-based kit that is capable of predicting responsiveness to therapy on a routine basis. To our knowledge, no monoclonal antibody against phospho-Y721 of c-KIT is currently available. This antibody will be used for a clinical trial designed to validate the correlation between the phosphorylated Y721 of c-KIT and the clinical response to imatinib in patients with acral lentiginous melanoma (Phase II application). Ultimately, a kit with this monoclonal antibody will be used as the primary tool to pre-screen patients for imatinib treatment.

Immunogen and Immunization: A peptide, CSDSTNEY[phospho]MDMKPG (SEQ ID NO: 1), will be synthesized. Five BALB/c mice (5 weeks old) will be used for immunogen injections. HPLC-purified peptides encompassing Y721 of c-KIT will be conjugated to Keyhole Limpet Hemocyanin (KLH). The peptide-KLH conjugates (50-100 μg) in an emulsion of Freund adjuvant will be injected intraperitoneally (IP) into mice. The peptides will be IP-injected three times. For boost before fusion, a final injection will be administered via tail vein intravenously (IV). At least 1 breeding pair of mice (male and female, nonsiblings, 8 weeks of age) are kept for breeding purpose to produce mice for monoclonal antibody production needs.

Blood Collection: Test bleeds of 0.01 ml will be taken from tail vein. Typical frequency of collection is once every two weeks. The blood will be collected into a suitable tube and allowed to clot at room temperature for up to 24 hours before the serum will be collected following centrifugation.

Termination: After the antiserum reaches the expected titer, mice will be sacrificed by carbon dioxide in glass desiccators from a connected closed container with dry ice, then cervical dislocation of the mouse to ensure death. The mouse is immersed in a beaker containing 70% ethanol prior to dissection. Using sterile forceps lift the thorax area and snip with sterile scissors. Peel skin over both sides to expose left side of the rib cage. Using another set of sterile forceps and scissors, remove the spleen from the left upper abdomen of the mouse. Afterwards, prepare the spleen cells for fusion.

Fusion: Spleen cells isolated from the selected animal will be fused with a myeloma cell line (SP2/0) to develop a hybridoma clone that secretes a single specific antibody. The fusion will be plated out and screened three times. The primary screening will be a direct ELISA against the immunogen to capture all positive fusion products. The positive fusion products will be scaled up and screened again. The secondary screening will differentiate between IgG and IgM clones, as well as verify positive fusion products are still producing antibody at the time of cryopreservation.

Subcloning: A monoclonal antibody-producing cell line will be isolated from a selected parental clone. The selected fusion clones will be subcloned by limiting dilution. Once colonies are established, the wells will be screened by a direct peptide-mediated ELISA assay. At least three clones from each parental clone will be selected for freeze down. Approximately five vials of each subclone will be stored.

Antibody Production: The monoclonal antibody will be collected through ascites or in vitro production for in vitro assay as described below. For cell lines developed in Balb/c mice, 5 Balb/c hybrid mice will be used to produce ascites for each clone. Ascite antibodies will be purified by Protein A or Protein G column to enrich IgGs.

In vitro Assays: A purified antibody with high titer identified by ELISA will tested in vitro using melanoma cell lines expressing c-KIT, e.g., MeWo, or c-KIT negative, e.g., WM-266-4 (ATCC # CRL-1676™). MeWo cells will be pre-treated with the c-KIT ligand, SCF, for 5-10 min before cell lysate will be harvested. MeWo cells without SCF stimulation and WM-266-4 cell lysates will serve as negative controls.

LSC analysis: Cells grown in chamber slides under different conditions as described above will be stained by anti-phospho-Y721 monoclonal antibodies followed by a secondary antibody conjugated with a fluorescent probe, e.g., Cy5. LSC analysis will be used to compare the fluorescence intensity of the stained cells. To increase sensitivity a chemiluminescent immunoassay (CLIA) may be used. The pre-immune serum from each animal will serves as a negative control as well. Similarly, antibodies will be tested on responder and non-responder tumor biopsies. The results will be compared with anti-phospho-c-KIT (Y721) polyclonal antibody.

Colorimetric analysis: Cells stained by the anti-phospho-Y721 of c-KIT monoclonal antibodies as described in the previous section will be incubated with goat anti-mouse immunoglobulins conjugated with horseradish peroxidase and its substrate, i.e., diaminobenzidine (DAB). The signal (brown color) will be observed under a light microscope.

Western Blot analysis: In addition, Western blot may be used to confirm the expression of phospho-c-KIT. Using anti-phospho-Y721 monoclonal antibody, levels of phospho-c-KIT (Y721) will be significantly higher in MeWo cells treated with SCF than that without SCF by LSC, colorimetric analysis, and Western blot. We expect to confirm more phosphorylation of c-KIT at Y721, ˜145 KD, in MeWo cells treated with SCF than that without SCF. No detectable phospho-c-KIT are expected in the negative control WM-266-4 cells. These assays will confirm results from the previous pilot studies and monoclonal anti-phospho-Y721 will have higher specificity in terms of signal-to-background ratio.

Protein A or Protein G purified antibodies may not yield antibodies with high specificity indicated by low titers determined by ELISA, colorimetric, and LSC analysis, and non-specific bands in the Western blot. If this occurs then peptide affinity columns will be employed to further purify the antibodies. Briefly, the antibodies will be passed through a column with non-phosphorylated peptide immunogen conjugated to the resin. This will allow non-specific antibodies to bind the column, the anti-phospho-Y721 antibodies will pass in the flow-through. The flow-through will then be passed through a second column with phospho-Y721 peptides conjugated to the resin. The anti-phospho-Y721 c-KIT antibodies may bind to the column allowing all non-binding materials to pass. Specific anti-phospho-Y721 will then be eluted off the column for the subsequent titer determination by ELISA, colorimetric, LSC, and Western blot analysis.

Once at least 3 clones that express monoclonal antibodies specific for phosphor-Y721-c-KIT with high titers as determined by ELISA (1:30,000), Western blot (1:1500), colorimetric (1:500), and LSC (1:500) are identified, a test kit may be developed for clinical use.

### Example 5

### In Vivo c-KIT Phosphorylation

Using in vitro assays of c-KIT phosphorylation, purified high-titer monoclonal antibody will bind to phosphorylated c-KIT. Co-labeling with an antibody to the c-KIT n-terminus allowed a quantitative analysis of c-KIT phosphorylation to total c-KIT concentration.

### Example 6

### Melanoma Test Kit

A Melanoma Test kit when available to the oncologists will identify melanoma patients who are likely to benefit by tyrosine phosphorylation inhibitor treatment. The kit will improve survival of melanoma patients by identifying patients with tumors that express hyperphosphorylation of c-KIT at Y721. Once approved as an In vitro Diagnostic (IVD) test, the kit will be highly valuable commercially and clinically. For example, the annual patient population with metastatic melanoma is estimated about 100,000. A clinical test to pre-screen patients with metastatic melanoma for imatinib treatment will be required for each patient to determine whether the patient will or will not response to imatinib treatment. As such, the c-KIT phosphorylation status in a patient's tumor as determined by the kit can be used to generate an individualized treatment plan that aims at maximizing the likelihood of response to imatinib and minimizing the side effect associated with the unnecessary treatment.

The c-KIT Cancer Response kit will optionally include:

1. Anti-phospho-Y721 of c-KIT monoclonal antibody

2. Goat anti-mouse antibody conjugated with horseradish peroxidase

3. DAB substrate buffer

4. Control slide contains sections of three pelleted, formalin-fixed, paraffin-embedded human melanoma cell lines: MeWo (+SCF), MeWo (−SCF), and WM-266-4, which represent the phospho-Y721 of c-KIT at high, basal, and background level, respectively.

5. Protocol of use.

Preliminary results have already shown technical feasibility of the proposed clinical assay and a strong commercial need for such an assay.

### Example 7

### Validation of Phospho-Tyrosine Screens

Validation of predicting response to therapy by measuring over expression of c-KIT Y721 will be conducted in two potential prospective clinical studies.

1) Biopsies will be obtained from untreated GIST patients and the tumors will be immunofluorescently stained with anti-c-KIT Y721 and Y703. Over expression of c-KIT Y721 will indicate enrollment for sunitinib therapy. Currently, the first line therapy for GIST treatment is Imatinib. Therefore, this study will have two arms; one arm will be Imatinib treated patients and the second arm will be c-KIT Y721 over expressing patients. The hazard ratios will be compared between the two arms to determine a significant difference in progression free survival and determine the value of using c-KIT Y721 as a predictive biomarker to select GIST patients for sunitinib vs. Imatinib therapy.

2) Acral lentiginous melanoma patients will be screened for over-expression of c-KIT Y721. If the patient is positive, then they will be enrolled on sunitinib and/or imatinib cancer treatment regimes to determine the correlation of c-KIT phospho-Y721 with clinical response to treatment regime.

These clinical studies will provide clinical monitoring regimes that track cancer response to treatment with c-KIT phosphorylation inhibitors such as imatinib and sunitinib treatment. A c-KIT diagnostic kit can be manufactured for patient screening using c-KIT phospho-Y721 antibodies with optional control samples, sample treatment buffer, sample storage materials, slide preparations and the like as well as instructions for sample preparation and assay. In another embodiment the diagnostic kit will consist of sample collection and storage materials allowing practitioners to collect patient samples and transmit stored samples to a central laboratory for testing. In one embodiment, sample collection and storage materials include blood sample tubes with premeasured buffer and inhibitors that maintain phosphorylation state of samples until the sample is assayed. Sample collection and storage materials may also include prepared biopsy needles with buffers and inhibitors that maintain phosphorylation state until the sample is assayed.

While the invention has been described with a limited number of embodiments, these specific embodiments are not intended to limit the scope of the invention as otherwise described and claimed herein. Modification and variations from the described embodiments exist. For instance, embodiments of the compositions described herein consist of or consist essentially of the enumerated components. Other embodiments are substantially free of or essentially free of any component not expressly recited. Some compositions substantially free of water while some compositions are substantially free of alcohol alkoxylates. Some compositions comprise less than 0.5 wt. percent of one or more alcohol alkoxylates. In some embodiments, the compositions may be substantially free of both water and alcohol alkoxylates. While the processes are described as comprising one or more steps, it should be understood that these steps may be practiced in any order or sequence unless otherwise indicated. These steps may be combined or separated.

Finally, any number disclosed herein should be construed to mean approximate, regardless of whether the word “about” or “approximate” is used in describing the number. Last but not the least, the claimed compositions are not limited to the processes described herein. They can be prepared by any suitable process. The appended claims intend to cover all such variations and modifications as falling within the scope of the invention.

### REFERENCES

All references are listed herein for the convenience of the reader, each is incorporated by reference in its entirety: Andersson, et al., “The complexity of KIT gene mutations and chromosome rearrangements and their clinical correlation in gastrointestinal stromal (pacemaker cell) tumors.” Am. J. Pathol. 160:15-22 (2002); Andre, et al., “Genomic organization of the human c-KIT gene: evolution of the receptor tyrosine kinase subclass III.” Oncogene 7:685-91 (1992); Arber, et al., “Paraffin section detection of the c-KIT gene product (CD117) in human tissues: value in the diagnosis of mast cell disorders.” Hum Pathol. 29:498-504 (1998); Duronio, et al., “p21ras activation via hemopoietin receptors and c-KIT requires tyrosine kinase activity but not tyrosine phosphorylation of p21ras GTPase-activating protein.” Proc. Natl. Acad. Sci. U.S.A. 89:1587-91 (1992); Strausberg, et al., “Generation and initial analysis of more than 15,000 full-length human and mouse cDNA sequences.” Proc. Natl. Acad. Sci. U.S.A. 99:16899-903 (2002); and Toyota, et al., “Complementary DNA cloning and characterization of truncated form of c-KIT in human colon carcinoma cells.” Cancer Res. 54:272-5 (1994).

What is claimed is:

1. An antibody that binds specifically to phosphorylated c-KIT, wherein said antibody binds a single epitope and does not bind to non-phosphorylated c-KIT.

2. The antibody of claim 1 wherein said antibody binds specifically to phosphorylated c-KIT at residue 703.

3. The antibody of claim 1 wherein said antibody binds specifically to phosphorylated c-KIT at residue 721.

4. The antibody of claim 1 wherein said antibody is monoclonal.

5. The antibody of claim 1 wherein said antibody is a recombinant single chain antibody.

6. A method of predicting cancer response to treatment comprising:
a) isolating a sample from a patient;
b) adding an antibody specific for phosphorylated c-KIT;
c) detecting binding of said antibody to phosphorylated c-KIT in said sample; and
d) identifying said patient as having a cancer responsive to treatment with an inhibitor of c-KIT phosphorylation.

7. The method of claim 6, wherein the sample is a blood sample or a cancer sample.

8. A method of monitoring patient response to cancer treatment comprising:
a) isolating a sample from a patient with a cancer;
b) adding an antibody specific for phosphorylated c-KIT;
c) detecting binding of said antibody to phosphorylated c-KIT in said sample;
d) repeating a), b), and c) after one or more treatments; and
e) determining response of said cancer to treatment
wherein a decrease in antibody binding indicates a remission of said cancer.

9. The method of claim 1, wherein said cancer is a solid tumor, blood based cancer, metastatic cancer, sarcoma, or melanoma.

10. The method of claim 9 wherein said cancer is a cancer selected from the group consisting of melanoma, gastrointestinal cancer, breast cancer, leukemia, lymphoma, lung cancer, acral lentiginous melanoma, renal cell carcinoma, colon carcinoma, small cell lung carcinoma, mast cell disease, testicular germ cell tumors, endometrial carcinomas, papillary and follicular thyroid carcinomas, small cell carcinomas, malignant melanomas, ovarian epithelial carcinomas, serous ovarian carcinoma, malignant melanoma, adenoid cystic carcinoma, salivary gland tumors, and metastatic renal cell carcinoma.

11. The method of claim 1, wherein said antibody binds specifically to phosphorylated c-KIT at residue 703.

12. The method of claim 1, wherein said antibody binds specifically to phosphorylated c-KIT at residue 721.

13. The method of claim 1, wherein said antibody is a monoclonal antibody.

14. The method of claim 1, further comprising: adding an antibody that binds to a non-phosphorylated epitope of c-KIT and comparing phosphorylated c-KIT to total c-KIT levels.

15. The method of claim 1, further comprising: measuring DNA fragmentation by TUNEL assay.

16. A kit for the detection of a cancer responsive to treatment comprising:
a) an antibody specific for phosphorylated c-KIT;
b) a detection antibody that binds said antibody specific for phosphorylated c-KIT;
c) a detection reagent that specifically reacts with the detection antibody; and
d) an optional control sample containing samples of known c-KIT phosphorylation.

17. The kit of claim 16, wherein said antibody specific for phosphorylated c-KIT binds specifically to phosphorylated residue 703.

18. The kit of claim 16, wherein said antibody specific for phosphorylated c-KIT binds specifically to phosphorylated residue 721.

19. The kit of claim 16, wherein the antibody specific for phosphorylated c-KIT is a monoclonal antibody.

20. The kit of claim 16, wherein the antibody specific for phosphorylated c-KIT is a recombinant single chain antibody.

21. The kit of claim 16, wherein said detection antibody is anti-mouse antibody conjugated with horseradish peroxidase and said detection reagent is DAB substrate buffer.

22. The kit of claim 16, wherein said detection antibody is anti-mouse antibody conjugated with a fluorescent probe and said detection reagent is a chemiluminescent immunoassay (CLIA).

23. The kit of claim 16, wherein said control sample is a slide containing high, basal, and background levels of phosphorylated c-KIT.
